(19) United States
(12) Patent Application Publication
(10) Pub. No.: US 2025/0111901 A1
(43) Pub. Date: Apr. 3, 2025

(54) DE NOVO GLYCOPEPTIDE SEQUENCING

(71) Applicant: VENN BIOSCIENCES CORPORATION, South San Francisco, CA (US)

(72) Inventors: Zhewei LIANG, San Carlos, CA (US); Mingqi LIU, San Carlos, CA (US)

(21) Appl. No.: 18/833,054

(22) PCT Filed: Feb. 14, 2023
(86) PCT No.: PCT/US2023/062542

§ 371 Date: Jul. 25, 2024

Related U.S. Application Data
(60) Provisional application No. 63/310,101, filed on Feb. 14, 2022.
(60) Provisional application No. 63/311,932, filed on Feb. 18, 2022.
(60) Provisional application No. 63/482,683, filed on Feb. 1, 2023.

(51) Int. Cl. G16B 40/10; G01N 30/86; G01N 30/88; G06N 3/044

(52) U.S. Cl. CPC G16B 40/10; G01N 30/8693; G01N 30/88; G06N 3/044

(57) ABSTRACT

A system and method of training a machine learning model to predict glycopeptide fragmentation patterns and retention times. Spectral data for a plurality of fragments of a glycopeptide structure is received. Glycan fragment composition data is generated using the spectral data. The glycan fragment composition data identifies a plurality of composition codes and a plurality of total intensities for a plurality of glycan fragments identified from the plurality of fragments using the spectral data. A linear glycan sequence is created using the glycan fragment composition data. A training input is formed for a machine learning model using the linear glycan sequence. The machine learning model is trained using the training input to predict a fragmentation pattern and a retention time for the glycopeptide structure.

### CROSS-REFERENCE TO RELATED APPLICATIONS

This application claims the priority of U.S. Provisional Patent Application Ser. No. 63/310,101 filed Feb. 14, 2022, 63/311,932, filed Feb. 18, 2022, and 63/482,683 filed Feb. 1, 2023, which are hereby all incorporated by reference herein in their entirety.

### FIELD

The present disclosure generally relates to methods and systems for analyzing, predicting, and creating sequences for non-linear peptide structures, such as glycopeptide structures. More particularly, the present disclosure relates to predicting and creating sequences for non-linear peptide structures in a format that can be used to train a machine learning model such as, for example, a deep learning model.

### BACKGROUND

Protein glycosylation and other post-translational modifications play vital roles in virtually all aspects of human physiology. Unsurprisingly, faulty or altered protein glycosylation often accompanies various disease states. The identification of aberrant glycosylation provides opportunities for early detection, intervention, and treatment of affected subjects. Current biomarker identification methods, such as those developed in the fields of proteomics and genomics, can be used to detect indicators of certain diseases, such as cancer, and to differentiate certain types of cancer from other, non-cancerous diseases. However, the use of glycoproteomic analyses has not previously been used to successfully identify disease processes.

Glycoprotein analysis is fraught with challenges on several levels, at least in part due to the non-linear structure or tree-type (e.g., branched) organization of glycans. For example, a single glycan composition in a peptide can contain a large number of isomeric structures due to different glycosidic linkages, branching patterns, and/or multiple monosaccharides having the same mass. In addition, the presence of multiple glycans that share the same peptide backbone can lead to assay signals from various glycoforms, lowering their individual abundances compared to aglycosylated peptides. Accordingly, the development of algorithms that can identify glycan structures on peptide fragments remains elusive. In light of the above, there is a need for improved analytical methods for accurately identifying and detecting non-linear structures, such as glycopeptides, based on the spectral data generated from mass spectrometry.

### BRIEF DESCRIPTION OF THE DRAWINGS

The present disclosure is described in conjunction with the appended figures:

FIG. 1 is a schematic diagram of an exemplary workflow 100 in accordance with one or more embodiments.

FIG. 2A is a schematic diagram of a preparation workflow in accordance with one or more embodiments.

FIG. 2B is a schematic diagram of data acquisition in accordance with one or more embodiments.

FIG. 2C is a schematic diagram of data acquisition 124 in accordance with one or more embodiments.

FIG. 3 is a block diagram of an analysis system in accordance with one or more embodiments.

FIG. 4 is a block diagram of a computer system in accordance with various embodiments.

FIG. 5 is a flowchart of a process for predicting glycopeptide fragmentation patterns and retention times in accordance with one or more embodiments.

FIG. 6 is a flowchart of a process for generating glycan composition data in accordance with one or more embodiments.

FIG. 7A is a flowchart of a process for creating a linear glycan sequence in accordance with one or more embodiments.

FIG. 7B is an illustration relating glycan composition data to linear fragment sequences in accordance with one or more embodiments.

FIG. 8 is a flowchart of a process for predicting glycopeptide fragmentation patterns and retention times in accordance with one or more embodiments.

FIG. 9 is a flowchart of a process for increasing accuracy and sensitivity in glycopeptide structure identification in accordance with one or more embodiments.

FIG. 10 is a flowchart of a process for increasing accuracy and sensitivity in N-linked glycopeptide structure identification in accordance with one or more embodiments.

FIG. 11 is a flowchart of a process for generating and targeting a panel of glycopeptide structures in accordance with one or more embodiments.

FIG. 12 is a flowchart of a process for powering O-linked glycopeptide structure discovery in accordance with one or more embodiments.

FIG. 13 is a flowchart of a process for enhancing DIA single shot mass spectrometry discovery and quantification in accordance with one or more embodiments.

FIGS. 14A-14D are illustrations of one example of how mass spectrometry data may be used to identify a linear glycan sequence in a format that is compatible with a machine learning model in accordance with one or more embodiments.

### DETAILED DESCRIPTION

### I. Overview

The embodiments described herein and in Appendix A recognize that glycoproteomics is an emerging field that can be used in the overall diagnosis and/or treatment of subjects with various types of diseases. Glycoproteomics aims to determine the positions, identities, and quantities of glycans and glycosylated proteins in a given sample (e.g., blood sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to diagnosing different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general due to several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

But to understand various disease conditions and to diagnose certain diseases, it may be important to perform analysis of glycoproteins and to identify not only the glycan but also the linking site (e.g., the amino acid residue of attachment) within the protein. Thus, there is a desire to provide a method for site-specific glycoprotein analysis to obtain detailed information about protein glycosylation patterns and to improve the accuracy and sensitivity with which glycopeptides can be detected, identified, and targeted via mass spectrometry.

Existing systems and methodologies are unable to provide information about the glycans of glycopeptide structures in a format that can be used with machine learning models. Accordingly, existing models may be unable to consider important glycan information. For example, existing methodologies do not allow for generating a two-dimensional matrix that can be input into a deep learning model to capture the non-linear structure (e.g., tree-like structure) of glycans. Recognizing, however, that machine learning can be used to improve the accuracy and sensitivity with which glycopeptides can be detected, identified, and targeted via mass spectrometry, the embodiments described herein provide various methods and systems for creating de novo linear glycan sequences for the glycans of glycopeptide structures. These linear glycan sequences may be in a format that is compatible with machine learning modeling. For example, these linear glycan sequences may be one-hot encoded for use with a deep learning model. Thus, the construction of these de novo linear glycan sequences solves a computer-related (or computer-specific) problem of how to capture important glycan information in a way that can be used to form input for a machine learning model.

The output of such machine learning modeling may be used in various applications that include, but are not limited to, augmenting a glycoproteomic database, enabling spectral matching and RT prediction for N-linked glycopeptide structures, expediting glycopeptide structure confirmation, generating new MRM-MS panels, powering O-linked glycopeptide structure discovery, facilitating DIA single-shot discovery and quantification, or a combination thereof. Thus, the embodiments described herein provide one or more improvements to the technical field of analyzing mass spectrometry data obtained for glycopeptides, the technical field of identifying and/or matching glycopeptides based on mass spectrometry data, the technical field of generating mass spectrometry panels, and the technical field of DIA single shot discovery and quantification, as well as other technical fields. The construction of de novo linear glycan sequences described herein may improve the overall performance power of mass spectrometry in the analysis of glycopeptides.

While the embodiments described herein are directed to creating linear glycan sequences for the glycans of glycopeptide structures, the methods and systems described herein may be applicable to other non-linear structures and are not limited to creating linear glycan sequences for the glycans of glycopeptide structures.

### II. Exemplary Descriptions of Terms

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “amino acid,” as used herein, generally refers to any organic compound that includes an amino group (e.g., —NH₂), a carboxyl group (—COOH), and a side chain group (R) which varies based on a specific amino acid. Amino acids can be linked using peptide bonds.

The term “alkylation,” as used herein, generally refers to the transfer of an alkyl group from one molecule to another. In various embodiments, alkylation is used to react with reduced cysteines to prevent the re-formation of disulfide bonds after reduction has been performed.

The term “linking site” or “glycosylation site” as used herein generally refers to the location where a sugar molecule of a glycan or glycan structure is directly bound (e.g., covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. Biological samples may include, but are not limited to synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, fecal material, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to blood and/or plasma. In some examples, biological samples include, but are not limited, to urine or stool. Biological samples include, but are not limited, to saliva. Biological samples include, but are not limited, to tissue dissections and tissue biopsies. Biological samples include, but are not limited, any derivative or fraction of the aforementioned biological samples. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a health state, a disease state). The term “biomarker” can be used interchangeably with the term “marker.”

The term “denaturation,” as used herein, generally refers to any molecule that loses quaternary structure, tertiary structure, and secondary structure which is present in their native state. Non-limiting examples include proteins or nucleic acids being exposed to an external compound or environmental condition such as acid, base, temperature, pressure, radiation, etc.

The term “denatured protein,” as used herein, generally refers to a protein that loses quaternary structure, tertiary structure, and secondary structure which is present in their native state.

The terms “digestion” or “enzymatic digestion,” as used herein, generally refers to a biological process that employs enzymes to break specific amino acid peptide bonds. For example, digesting a peptide includes contacting the peptide with a digesting enzyme, e.g., trypsin to produce fragments of the glycopeptide. In some examples, a protease enzyme is used to digest a glycopeptide. The term “protease” refers to an enzyme that performs proteolysis or breakdown of large peptides into smaller polypeptides or individual amino acids. Examples of a protease include, but are not limited to, one or more of a serine protease, threonine protease, cysteine protease, aspartate protease, glutamic acid protease, metalloprotease, asparagine peptide lyase, and any combinations of the foregoing. Enzymatic digestion may be used in preparation for mass spectrometry using trypsin digestion protocols. Proteins may be digested using other proteases in preparation for mass spectrometry if access is limited to cleavage sites.

The term “disease state” as used herein, generally refers to a condition that affects the structure or function of an organism. Non-limiting examples of causes of disease states may include pathogens, immune system dysfunctions, cell damage caused by aging, cell damage caused by other factors (e.g., trauma and cancer). Disease states can include any state of a disease whether symptomatic or asymptomatic. Disease states can include disease stages of a disease progression. Disease states can cause minor, moderate, or severe disruptions in structure or function of an organism (e.g., a subject).

The term “fragment” or “fragmentation product,” as used herein, generally refers to the product of an ion fragmentation process which occurs using a mass spectrometry instrument or system (e.g., an MRM-MS instrument and/or discovery/untargeted MS instrument). For example, a fragment may result from digestion of an amino acid sequence (e.g., a protein, glycoprotein, peptide, or glycopeptide) that subsequently undergoes mass spectrometry analysis.

A mass spectrometry fragmentation process may result in one or more fragmentation products. A fragmentation product may relate to a pre-identified target. A fragmentation product may relate to a previously unidentified product (e.g., resulting from discovery/untargeted MS). Fragmenting may produce various fragments having the same mass but varying charges. Thus, some fragments having the same mass may have different product m/z ratios. A biomarker, such as one or more of the biomarkers described herein, may produce more than one product m/z. A fragment of a glycopeptide (glycosylated peptide) may be referred to as a “glycopeptide fragment” or a “glycosylated peptide fragment.” Unless specified otherwise, within the specification, “glycopeptide fragments” or “fragments of a glycopeptide” refer to the fragments produced directly by using a mass spectrometer optionally after the glycoprotein has been digested enzymatically to produce the glycopeptides.

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycopeptide” or “glycopolypeptide” as used herein, generally refers to a peptide or polypeptide comprising at least one glycan residue. In various embodiments, glycopeptides comprise carbohydrate moieties (e.g., one or more glycans) covalently attached to a side chain (i.e. R group) of an amino acid residue.

The term “glycopeptide” as used herein, generally refers to a glycosylated peptide (or glycopeptide) having an amino acid sequence that is the same as part (but not all) of an amino acid sequence of a glycosylated protein. A glycosylated protein may be digested to generate one or more glycopeptides. A glycosylated peptide (or glycopeptide) having an amino acid sequence that is the same as part (but not all) of the amino acid sequence of the glycosylated protein glycosylated peptide may undergo ion fragmentation within a mass spectrometry instrument (e.g., an MRM-MS instrument). MRM refers to multiple-reaction-monitoring.

The term “glycoprotein,” as used herein, generally refers to a protein having at least one glycan residue bonded thereto. In some examples, a glycoprotein is a protein with at least one oligosaccharide chain covalently bonded thereto. Examples of glycoproteins include but are not limited to the peptide structures including glycan molecules shown in the various Tables presented herein.

The term “liquid chromatography,” as used herein, generally refers to a technique used to separate a sample into parts. Liquid chromatography can be used to separate, identify, and quantify components.

The term “mass spectrometry,” as used herein, generally refers to an analytical technique used to identify molecules. In various embodiments described herein, mass spectrometry can be involved in characterization and sequencing of proteins.

The term “m/z” or “mass-to-charge ratio,” as used herein, generally refers to an output value from a mass spectrometry instrument. In various embodiments, m/z can represent a relationship between the mass of a given ion and the number of elementary charges that it carries. The “m” in m/z stands for mass and the “z” stands for charge. In some embodiments, m/z can be displayed on an x-axis of a mass spectrum.

The term “patient,” as used herein, generally refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.” As used herein, the phrase “peptide,” is meant to include glycopeptides unless stated otherwise.

The terms “protein” or “polypeptide” or “peptide” may be used interchangeably herein and generally refer to a molecule including at least three amino acid residues. Proteins can include polymer chains made of amino acid sequences linked together by peptide bonds. Proteins may be digested in preparation for mass spectrometry using trypsin digestion protocols. Proteins may be digested using other proteases in preparation for mass spectrometry if access is limited to cleavage sites.

The term “peptide structure,” as used herein, generally refers to peptides or a portion thereof or glycopeptides or a portion thereof. In various embodiments described herein, a peptide structure can include any molecule comprising at least two amino acids in sequence. A “glycopeptide structure” may be one example of a peptide structure. A “glycopeptide structure” may be a glycopeptide or a portion thereof.

The term “reduction,” as used herein, generally refers to the gain of an electron by a substance. In various embodiments described herein, a sugar can directly bind to a protein, thereby, reducing the amino acid to which it binds. Such reducing reactions can occur in glycosylation. In various embodiments, reduction may be used to break disulfide bonds between two cysteines.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cₘ (H₂O)ₙ).

The term “subject,” as used herein, generally refers to an animal, such as a mammal (e.g., human) or avian (e.g., bird), or other organism, such as a plant. For example, the subject can include a vertebrate, a mammal, a rodent (e.g., a mouse), a primate, a simian, or a human. Animals may include, but are not limited to, farm animals, sport animals, and pets. A subject can include a healthy or asymptomatic individual, an individual that has or is suspected of having a disease (e.g., cancer) or a pre-disposition to the disease, and/or an individual that needs therapy or is suspected of needing therapy. A subject can be a patient. A subject can include a microorganism or microbe (e.g., bacteria, fungi, archaea, viruses). However, in the context of diagnosing ovarian cancer, the subject is female unless explicitly specified otherwise. A subject may be one who has been previously identified as having a disease or a condition, and optionally has already undergone, or is undergoing, a therapeutic intervention for the disease or condition. Alternatively, a subject can also be one who has not been previously diagnosed as having a disease or a condition. For example, a subject can be one who exhibits one or more risk factors for a disease or a condition, or a subject who does not exhibit disease risk factors, or a subject who is asymptomatic for a disease or a condition. A subject can also be one who is suffering from or at risk of developing a disease or a condition.

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, one or more deep learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model. In one or more embodiments, a machine learning model may be built using any number or combination of the algorithms or models described above. For example, a machine learning model may include a deep learning model. The deep learning model may include any number of or combination of deep learning algorithms, neural networks, or other representational learning algorithms involving multiple layers. A deep learning model may include, for example, a supervised learning model or algorithm, a semi-supervised learning model or algorithm, an unsupervised learning model or algorithm, or a combination thereof.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “target glycopeptide analyte,” may refer to a peptide structure (e.g., glycosylated or aglycosylated/non-glycosylated), a fraction of a peptide structure, a sub-structure (e.g., a glycan or a glycosylation site) of a peptide structure, a product of one or more of the above listed structures and sub-structures, associated detection molecules (e.g., signal molecule, label, or tag), or an amino acid sequence that can be measured by mass spectrometry.

As used herein, a “peptide data set,” may be used interchangeably with “peptide structure data” and can refer to any data of or relating to a peptide from a resulting mass spectrometry run. A peptide data set can comprise data obtained from a sample or biological sample using mass spectrometry. A peptide dataset can comprise data relating to an external standard, data relating to an internal standard, and data relating to a target glycopeptide analyte of a sample. A peptide data set can result from analysis originating from a single run. In some embodiments, the peptide data set can include raw abundance and mass to charge ratios for one or more peptides.

As used herein, a “a transition,” may refer to or identify a peptide structure. In some embodiments, a transition can refer to the specific pair of m/z values associated with a precursor ion and a product or fragment ion.

As used herein, a “non-glycosylated endogenous peptide” (“NGEP”) may refer to a peptide structure that does not comprise a glycan molecule. In various embodiments, an NGEP and a target glycopeptide analyte can originate from the same subject. In various embodiments, an NGEP and a target glycopeptide analyte may be derived from the same protein sequence. In some embodiments, the NGEP and the target glycopeptide analyte may be derived from or include the same peptide sequence. In various embodiments, an NGEP can be labeled with an isotope in preparation for mass spectrometry analysis.

As used herein, “abundance,” may refer to a quantitative value generated using mass spectrometry. In various embodiments, the quantitative value may relate to the amount of a particular peptide structure. In some embodiments, the quantitative value may comprise an amount of an ion produced using mass spectrometry. In some embodiments, the quantitative value may be expressed as an m/z value. In other embodiments, the quantitative value may be expressed in atomic mass units.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot.

As used herein, an “internal standard,” may refer to something that can be contained (e.g., spiked-in) in the same sample as a target glycopeptide analyte undergoing mass spectrometry analysis. Internal standards can be used for calibration purposes. Additionally, internal standards can be used in the systems and method described herein. In some aspects, an internal standard can be selected based on similarity m/z and or retention times and can be a “surrogate” if a specific standard is too costly or unavailable. Internal standards can be heavy labeled or non-heavy labeled.

The term “data-dependent acquisition (DDA) mass spectrometry” as used herein, may generally refer to one or more methods of molecular structure determination in which a fixed number of precursor ions (e.g., ions of a narrow m/z range such as, but not limited to, 1 or 2 m/z) from a first stage of tandem mass spectrometry are selected and analyzed in a second stage of tandem mass spectrometry. Thus, the fragments resulting from the second stage of tandem mass spectrometry may be for a few target analytes (e.g., one to three target analytes, one to three target analytes, etc.).

The term “data-independent acquisition (DIA) mass spectrometry” as used herein, may generally refer to one or more methods of molecular structure determination in which all precursor ions within a selected m/z range (e.g., an m/z range such as, but not limited to, 10 or 20 m/z) from a first stage of tandem mass spectrometry are fragmented and analyzed in a second stage of tandem mass spectrometry. Tandem mass spectra may be acquired either by fragmenting all ions that enter the mass spectrometer at a given time (called broadband DIA) or by sequentially isolating and fragmenting ranges of m/z. As such, in DIA mass spectrometry, the direct relation between a precursor ion and its fragment ions may be lost. Because multiple peptides may likely fall within the m/z range a resulting spectral profile may be complex and require deconvolution. An advantage to DIA mass spectrometry may be the ability to quantify peptides without needing to predefine peptides of interest.

The term “untargeted mass spectrometry” as used herein, may generally refer to a mass spectrometry method allowing interrogation of a broad range of peptide structures without the requirement of predefining peptides of interest. Untargeted data acquisition allows for a mass spectrometry system to parse unknown precursor ions for collision-induced dissociation and collect product ion spectra without a prior knowledge of the target being analyzed. Untargeted mass spectrometry may apply universal sample preparation techniques, combinations of chromatographical separation techniques that target different chemical groups of analytes, and a combination of full-scan MS detection and data-dependent fragmentation analysis enabling large amounts of data to be generated. Because multiple peptides may likely fall within the m/z range a resulting spectral profile may be complex and require deconvolution.

The term “untargeted mass spectrometry system” as used herein, may generally refer to a mass spectrometry system capable of carrying out untargeted mass spectrometry methods (e.g., collection of product ion spectra without a prior knowledge of the target being analyzed and then the ability to deconvolute the resulting spectral data). A non-limiting example of such a system may include the Orbitrap Exploris™ 480 Mass Spectrometer combined with use of hydrophilic interaction chromatography (HILIC) systems. HILIC may be especially well suited for separation of peptide structures based on polarity. Some untargeted mass spectrometry systems may include a use of miniaturized of a high-pressure liquid chromatography (HPLC) system where peptide structures may be separated in capillary columns with relatively thin diameters (e.g., <100 μm).

The term “shotgun proteomics” as used herein, may generally refer to the use of proteomics techniques to identify proteins in complex mixtures using a combination of high-performance liquid chromatography combined with mass spectrometry. A non-limiting example of an approach to shotgun proteomics may include starting with the proteins in a mixture being digested and the resulting peptides are separated by liquid chromatography. Tandem mass spectrometry may then be used to identify the peptides.

### III. Overview of Exemplary Workflow

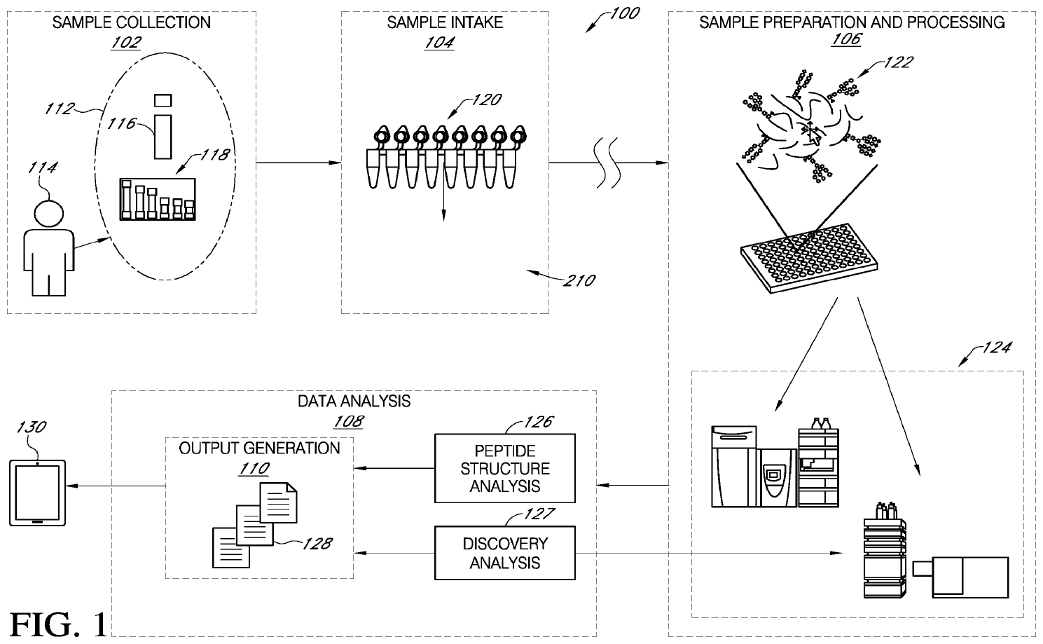

FIG. 1 is a schematic diagram of an exemplary workflow 100 in accordance with one or more embodiments. Workflow 100 may include various operations including, for example, sample collection 102, sample intake 104, sample preparation and processing 106, data analysis 108, and output generation 110.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be obtained in any of a number of different ways. In various embodiments, biological sample 112 includes whole blood sample 116 obtained via a blood draw. In other embodiments, biological sample 112 includes set of aliquoted samples 118 that includes, for example, a serum sample, a plasma sample, a blood cell (e.g., white blood cell (WBC), red blood cell (RBC) sample, another type of sample, or a combination thereof. Biological samples 112 may include nucleotides (e.g., ssDNA, dsDNA, RNA), organelles, amino acids, peptides, proteins, carbohydrates, glycoproteins, or any combination thereof.

In various embodiments, a single run can analyze a sample (e.g., the sample including a peptide analyte), an external standard (e.g., an NGEP of a serum sample), and an internal standard. As such, abundance or raw abundance for the external standard, the internal standard, and target glycopeptide analyte can be determined by mass spectrometry in the same run.

In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein. In additional embodiments, blank samples may be processed to prevent column fouling.

Sample intake 104 may include one or more various operations such as, for example, aliquoting, registering, processing, storing, thawing, and/or other types of operations. In one or more embodiments, when biological sample 112 includes whole blood sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system. In one or more embodiments, data acquisition 124 may include use of, for example, an untargeted mass spectrometry system to aid in the discovery of glycopeptide structures.

Data analysis 108 may include, for example, peptide structure analysis 126, discovery analysis 127, or both. Discovery analysis 127 may include analyzing spectral data generated via data acquisition 124 to identify peptide sequences and glycan sequences associated with glycopeptide structures. In particular, the glycan sequences identified may be linear glycan sequences that are presented in a format that can be used to form an input for a machine learning model.

In some embodiments, data analysis 108 also includes output generation 110. In other embodiments, output generation 110 may be considered a separate operation from data analysis 108. Output generation 110 may include, for example, generating final output 128 based on the results of peptide structure analysis 126. Final output 128 may be used for determining research, diagnosis, and/or treatment.

In various embodiments, final output 128 is comprised of one or more outputs. Final output 128 may take various forms. For example, final output 128 may be a report that includes, for example, a diagnosis output, a treatment output (e.g., a treatment design output, a treatment plan output, or combination thereof), analyzed data (e.g., relativized and normalized) or combination thereof. In some embodiments, report can comprise a target glycopeptide analyte concentration as a function of the NGEP concentration value and the normalized abundance. In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). Accordingly, workflow 100 may be implemented in any of a number of different ways for use in the discovery and sequencing of glycopeptide structures and/or the research, diagnosis, and/or treatment of one or more disease states.

### IV. Detection and Quantification of Peptide Structures

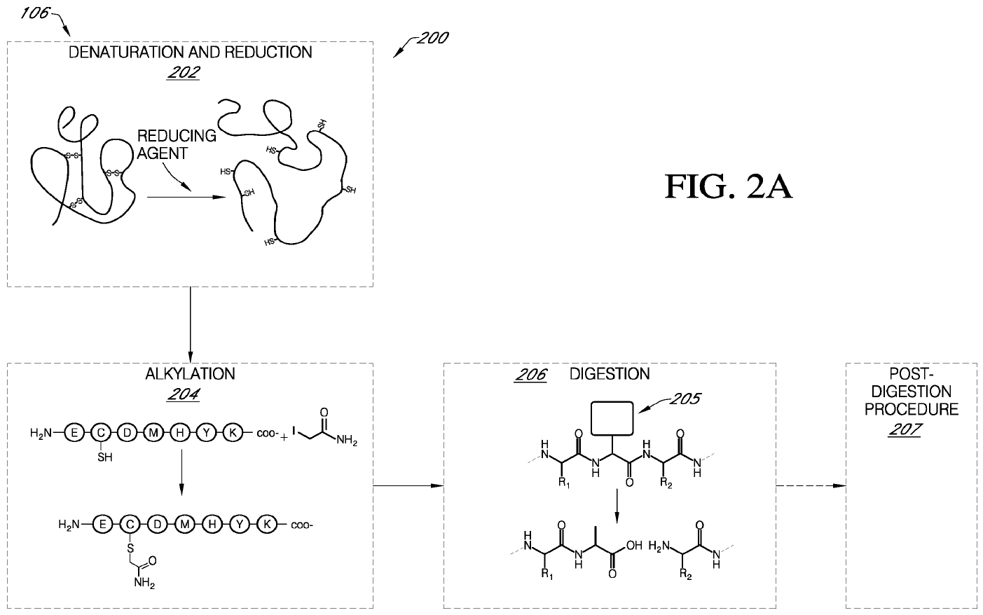

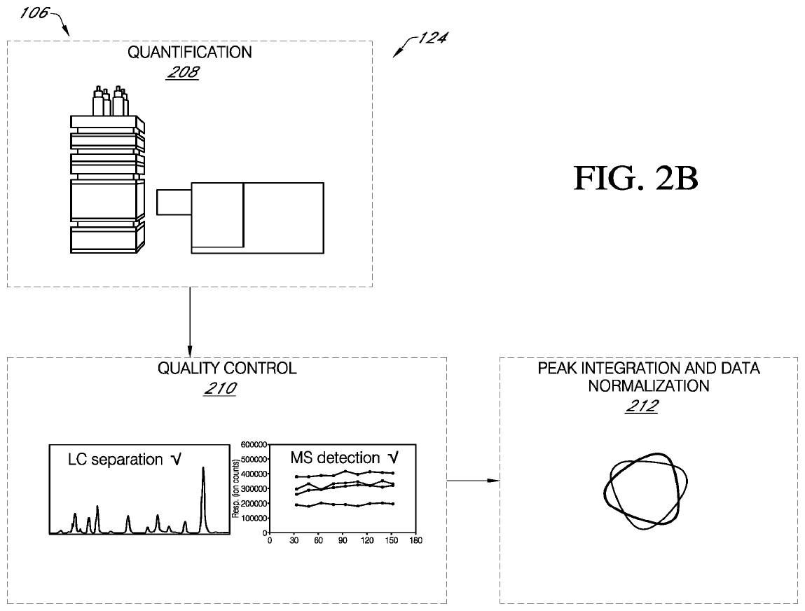

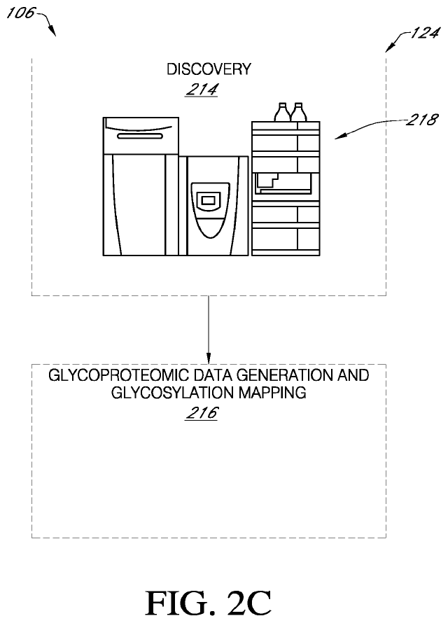

FIGS. 2A and 2B are schematic diagrams of a workflow for sample preparation and processing 106 in accordance with one or more embodiments. FIGS. 2A and 2B are described with continuing reference to FIG. 1. Sample preparation and processing 106 may include, for example, preparation workflow 200 shown in FIG. 2A and data acquisition 124 shown in FIG. 2B.

### IV.A. Sample Preparation and Processing

FIG. 2A is a schematic diagram of preparation workflow 200 in accordance with one or more embodiments. Preparation workflow 200 may be used to prepare a sample, such as a sample of set of samples 120 in FIG. 1, for analysis via data acquisition 124. For example, this analysis may be performed via mass spectrometry (e.g., LC-MS). In various embodiments, preparation workflow 200 may include denaturation and reduction 202, alkylation 204, and digestion 206. All areas of the preparation workflow can cause inconsistency between different samples and different experiments, necessitating, the improved normalization systems and methods described herein and throughout.

In general, polymers, such as proteins, in their native form, can fold to include secondary, tertiary, and/or other higher order structures. Such higher order structures may functionalize proteins to complete tasks (e.g., enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g., peptide/protein molecules) may be desired to obtain sequence information. In some embodiments, unfolding a polymer may include denaturing the polymer, which may include, for example, linearizing the polymer.

In one or more embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 1). Denaturation and reduction 202 includes, for example, a denaturation procedure and a reduction procedure. In some embodiments, the denaturation procedure may be performed using, for example, thermal denaturation, where heat is used as a denaturing agent. The thermal denaturation can disrupt ionic bonding, hydrophobic interactions, and/or hydrogen bonding.

In various embodiments, the denaturation procedure may include using one or more denaturing agents. In one or more embodiments, the denaturation procedure may include using temperature. In one or more embodiments, the denaturation procedure may include using one or more denaturing agents in combination with heat. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. In various embodiments, a reducing agent can produce an alkaline pH. A reducing agent may take the form of, for example, without limitation, dithiothreitol (DTT), tris(2-carboxyethyl) phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used to add an acetamide group to a sulfur on each cysteine residue to prevent disulfide linkages from reforming. In various embodiments, an acetamide group can be added by reacting one or more alkylating agents with a reduced protein. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated proteins formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of the lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In one or more embodiments, trypsin is used to digest serum samples. In one or more embodiments, trypsin/LysC cocktails are used to digest plasma samples.

In some embodiments, digestion 206 further includes a quenching procedure. The quenching procedure may be performed by acidifying the sample (e.g., to a pH<3). In some embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards.

Although preparation workflow 200 has been described with respect to a sample created or taken from biological sample 112 that is blood-based (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptides structures 122.

### IV.B. Peptide Structure Identification and Quantitation

FIG. 2B is a schematic diagram of data acquisition 124 in accordance with one or more embodiments. In various embodiments, data acquisition 124 can commence following sample preparation 200 described in FIG. 2A. In various embodiments, data acquisition 124 can comprise quantification 208, quality control 210, and peak integration and normalization 212.

In various embodiments, targeted quantification 208 of peptides and glycopeptides can incorporate use of liquid chromatography-mass spectrometry LC/MS instrumentation. For example, LC-MS/MS, or tandem MS may be used. In general, LC/MS (e.g., LC-MS/MS) can combine the physical separation capabilities of liquid chromatograph (LC) with the mass analysis capabilities of mass spectrometry (MS). According to some embodiments described herein, this technique allows for the separation of digested peptides to be fed from the LC column into the MS ion source through an interface.

In various embodiments, any LC/MS device can be incorporated into the workflow described herein. In various embodiments, an instrument or instrument system suited for identification and targeted quantification 208 may include, for example, a Triple Quadrupole LC/MS™ (QQQ LC/MS). In various embodiments, targeted quantification 208 is performed using multiple reaction monitoring mass spectrometry (MRM-MS).

In various embodiments described herein, identification of a particular protein or peptide and an associated quantity can be assessed. In various embodiments described herein, identification of a particular glycan and an associated quantity can be assessed. In various embodiments described herein, particular glycans can be matched to a glycosylation site on a protein or peptide and the abundances measured.

In some cases, targeted quantification 208 includes using a specific collision energy associated for the appropriate fragmentation to consistently see an abundant product ion. Glycopeptide structures may have a lower collision energy than aglycosylated peptide structures. When analyzing a sample that includes glycopeptide structures, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

### IV.C. Discovery of Glycopeptide

FIG. 2C is a schematic diagram of data acquisition 124 in accordance with one or more embodiments. In various embodiments, data acquisition 124 can commence following sample preparation 200 described in FIG. 2A. In various embodiments, data acquisition 124 can comprise discovery 214 and glycoproteomic data generation and glycosylation mapping 216. In one or more embodiments, discovery 214 is performed using an untargeted mass spectrometry system 218. The output of untargeted mass spectrometry system 218 may be used in glycoproteomic data generation and glycosylation mapping 216 to form spectral data that is then used for data analysis 108 in FIG. 1. The spectral data may include mass spectrometry data that can be used to build a glycopeptide spectral library or may include the glycopeptide spectral library itself.

The glycopeptide spectral library may identify, for example, for a detected glycopeptide structure, an identified peptide sequence for the glycopeptide structure; an observed retention time for the glycopeptide structure; an identified glycan composition for the glycopeptide structure; and a mass, a charge, and an intensity for each fragment of a plurality of fragments detected for the glycopeptide structure. For those fragments that are Y ions (e.g., glycan fragments), the glycopeptide spectral library further identifies a glycan composition. The glycopeptide spectral library may list the plurality of fragments in increasing order with respect to mass.

In other embodiments, the glycopeptide spectral library may include a subset of the information described above, additional information as compared to that described above, or a combination thereof. For example, in one or more embodiments, the glycopeptide spectral library may identify, for example, for a detected glycopeptide structure, an identified peptide sequence for the glycopeptide structure; an observed retention time for the glycopeptide structure; an identified glycan composition for the glycopeptide structure; and Y ion information (e.g., a mass, a charge, an intensity, and a glycan composition for each Y ion or glycan fragment). In other words, the glycopeptide spectral library may, in some cases, not include any information regarding y ions or b ions.

### V. Glycopeptide Sequencing

### V.A. Exemplary System for Glycopeptide Sequencing

### V.A.1. System for Constructing Glycopeptide Sequences

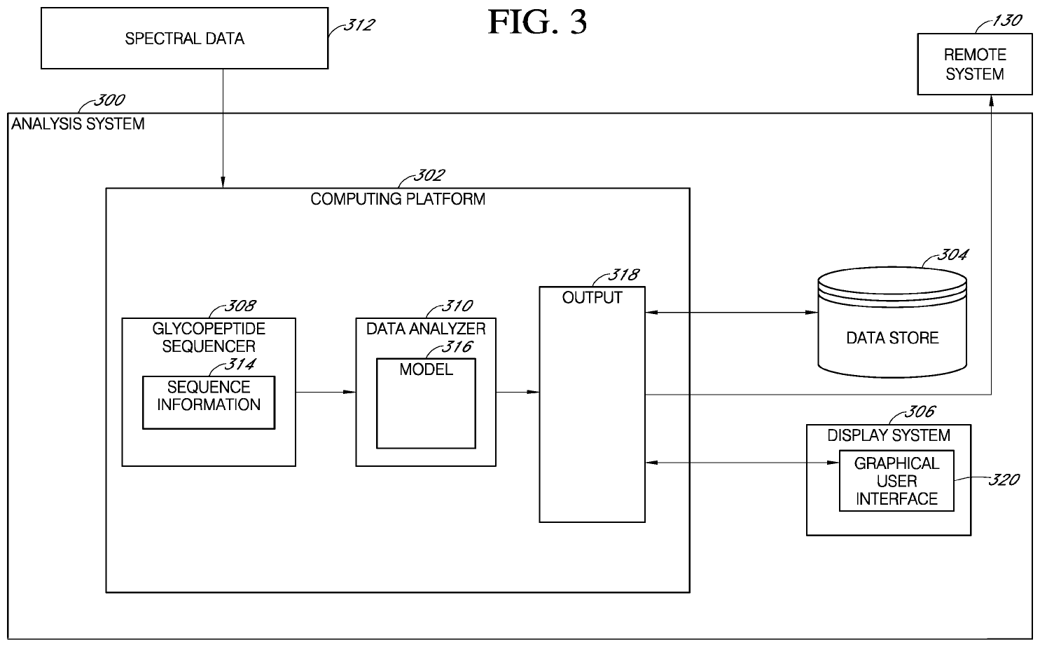

FIG. 3 is a block diagram of an analysis system 300 in accordance with one or more embodiments. Analysis system 300 may be used to perform various operations including, but not limited to, detecting and analyzing peptide structures that have been associated with one or more disease states, constructing de novo glycopeptide sequences, and/or one or more other operations. Analysis system 300 is one example of an implementation for a system that may be used to perform data analysis 108 in FIG. 1. For example, analysis system 300 may be used to perform peptide structure analysis 126 in FIG. 1, discovery analysis 127 in FIG. 1, or both. Thus, analysis system 300 is described with continuing reference to workflow 100 as described in FIGS. 1, 2A, 2B, and/or 2C.

Analysis system 300 may include computing platform 302 and data store 304. In some embodiments, analysis system 300 also includes display system 306. Computing platform 302 may take various forms. In one or more embodiments, computing platform 302 includes a single computer (or computer system) or multiple computers in communication with each other. In other examples, computing platform 302 takes the form of a cloud computing platform.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

In one or more embodiments, analysis system 300 includes, for example, glycopeptide sequencer 308 and data analyzer 310, each which may be implemented using hardware, software, firmware, or a combination thereof. In one or more embodiments, glycopeptide sequencer 308 and data analyzer 310 are implemented using computing platform 302.

Glycopeptide sequencer 308 receives spectral data 312 for processing. Spectral data 312 may include, for example, mass spectrometry data obtained via untargeted or non-targeted mass spectrometry. Spectral data 312 may be, for example, the spectral data that is output from sample preparation and processing 106 in FIGS. 1, 2A, 2B, and/or 2C. For example, spectral data 310 may be the spectral data that is output from discovery 214 in FIG. 2C.

In one or more embodiments, spectral data 312 may include a glycopeptide spectral library or the mass spectrometry data in spectral data 312 may be used to build a glycopeptide spectral library. The glycopeptide spectral library may identify, for example, for a detected glycopeptide structure, a peptide sequence for the glycopeptide structure; an observed retention time for the glycopeptide structure; an observed glycan composition for the glycopeptide structure; and a mass, a charge, and an intensity for each fragment of a plurality of fragments detected for the glycopeptide structure. For those fragments that are Y ions (e.g., glycan fragments), the glycopeptide spectral library further identifies a glycan composition. The glycopeptide spectral library may list the plurality of fragments in increasing order with respect to mass.

Glycopeptide sequencer 308 uses spectral data 312 to generate sequence information 314. Sequence information 314 may include, for example, a peptide sequence and a linear glycan sequence for at least one glycopeptide structure. The linear glycan sequence may be a “theoretical,” constructed sequence that is in a format that is compatible with machine learning.

Sequence information 314 may be used by data analyzer 310. Data analyzer 310 may include, for example, model 316. Data analyzer 410 can form a training input for model 316 using sequence information 314. Model 316 includes a machine learning model that can be trained using this training input. In one or more embodiments, model 316 includes a deep learning model. Model 316 may be trained to predict output 318. Output 318 may include, for example, without limitation, a fragmentation pattern and a retention time for at least one glycopeptide structure. The fragmentation pattern may include a set of m/z ratios, a set of intensities, or both for the glycopeptide structure. The retention time may be, for example, an index retention time (iRT).

Output 318 may be stored in data store 304 and/or sent to remote system 130 for processing in some examples. In other embodiments, output 318 may be displayed on graphical user interface 320 in display system 306 for viewing by a human operator.

Output 318 may be use in various applications that include, but are not limited to, augmenting a glycoproteomic database, enabling spectral matching and RT prediction for N-linked glycopeptide structures, expediting glycopeptide structure confirmation, generating new MRM-MS panels, powering O-linked glycopeptide structure discovery, facilitating DIA single-shot discovery and quantification, or a combination thereof.

### V.A.2. Computer Implemented System

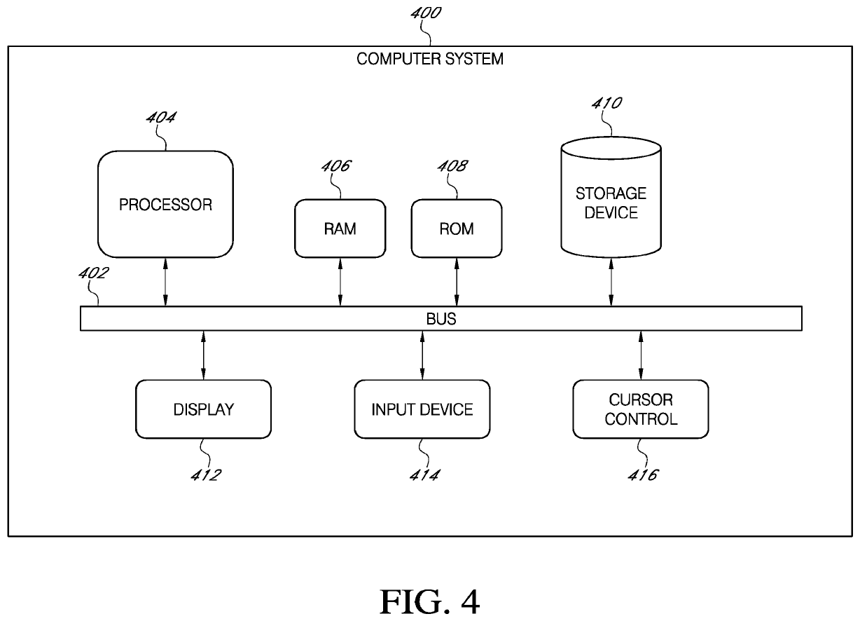

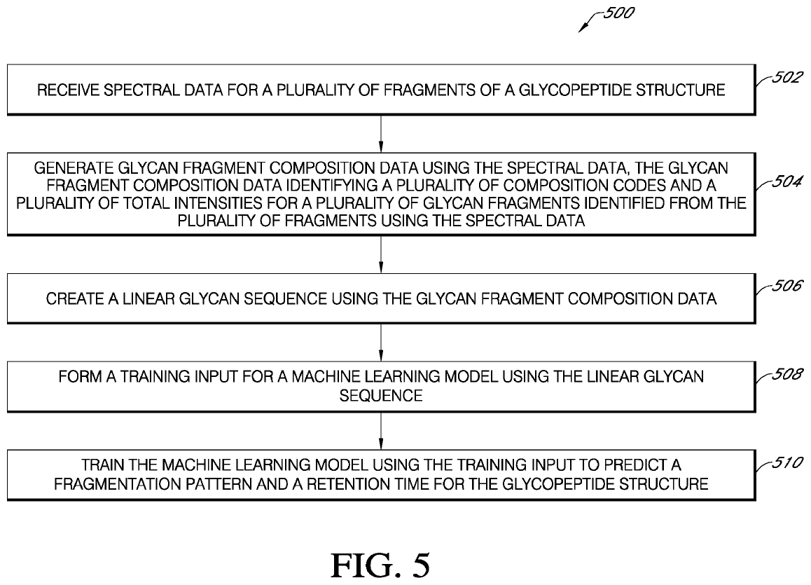

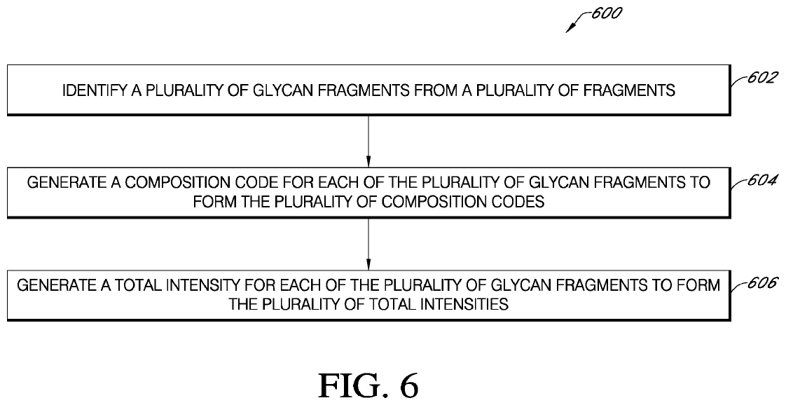

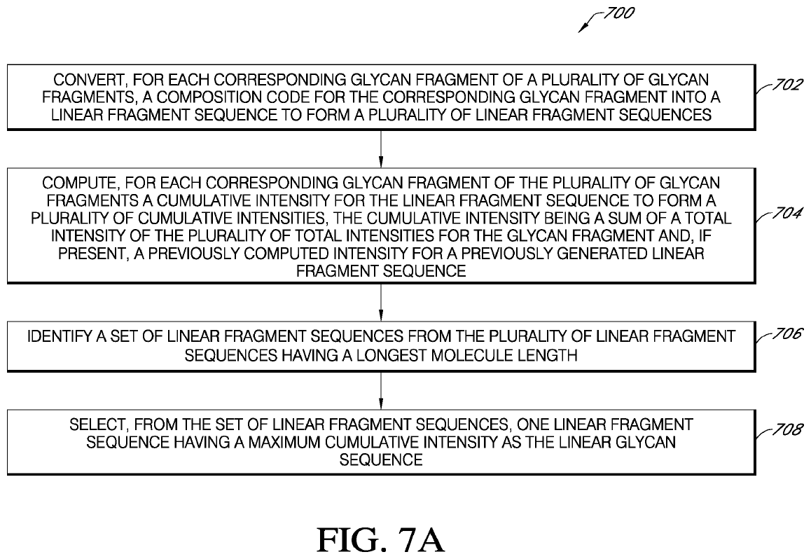

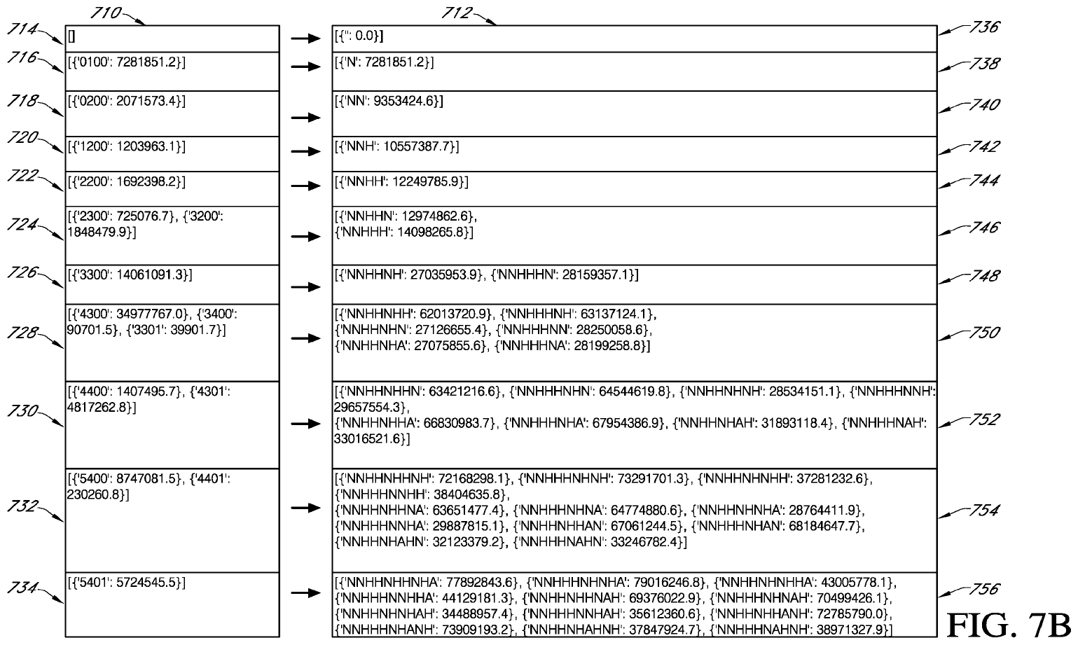

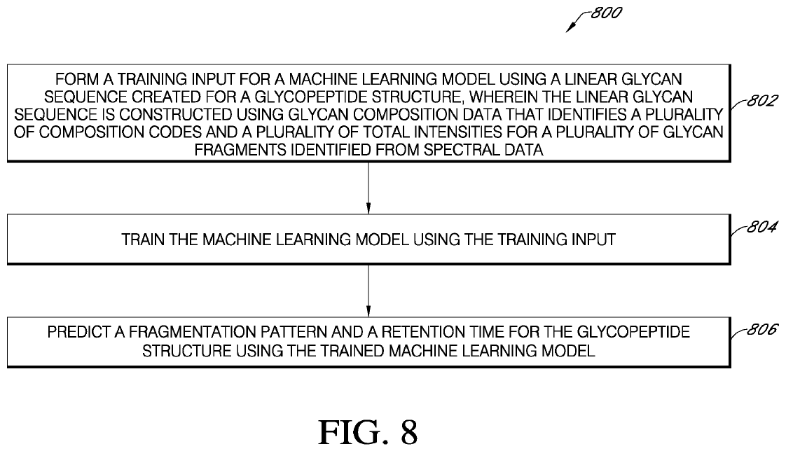

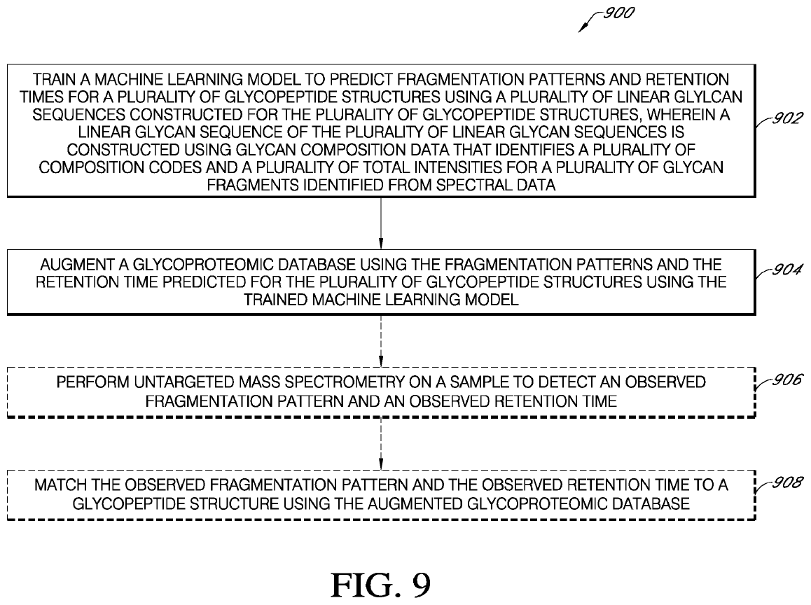

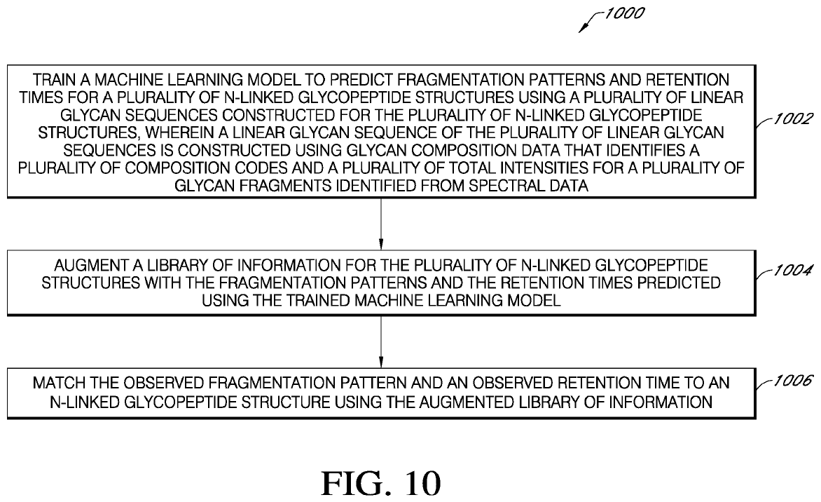

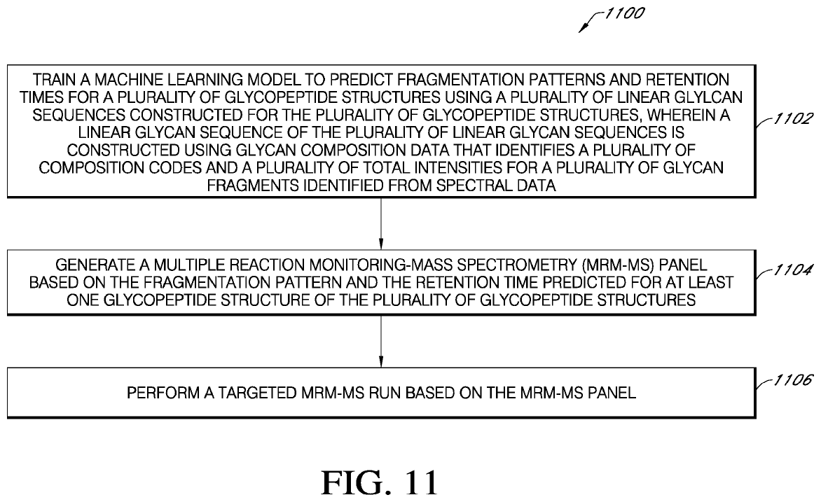

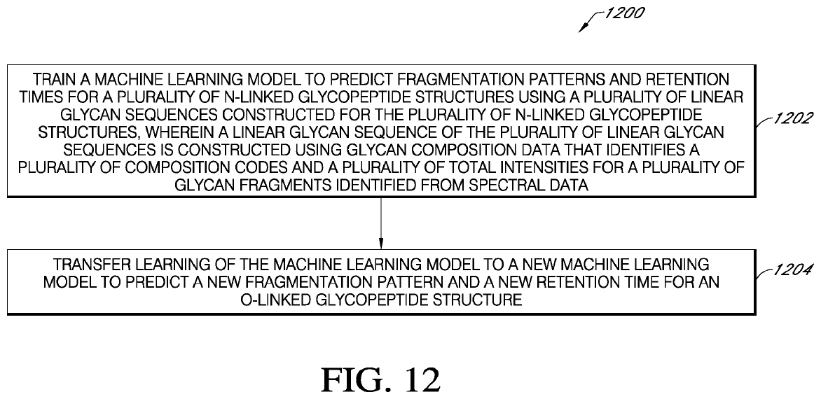

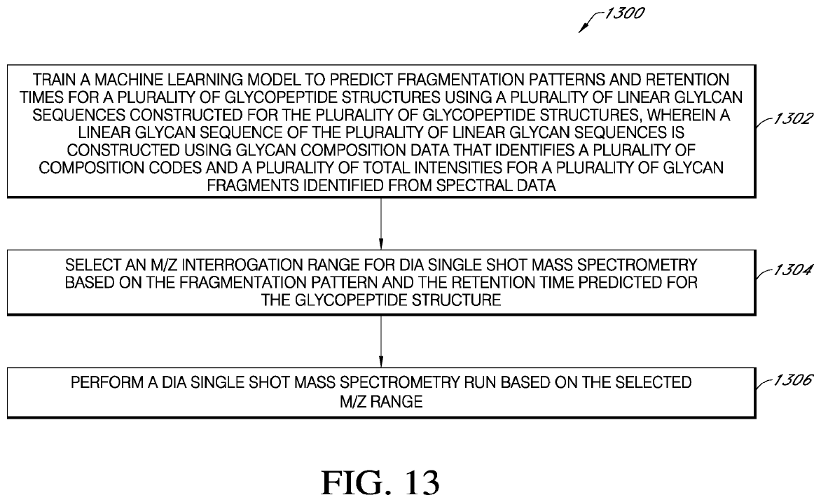

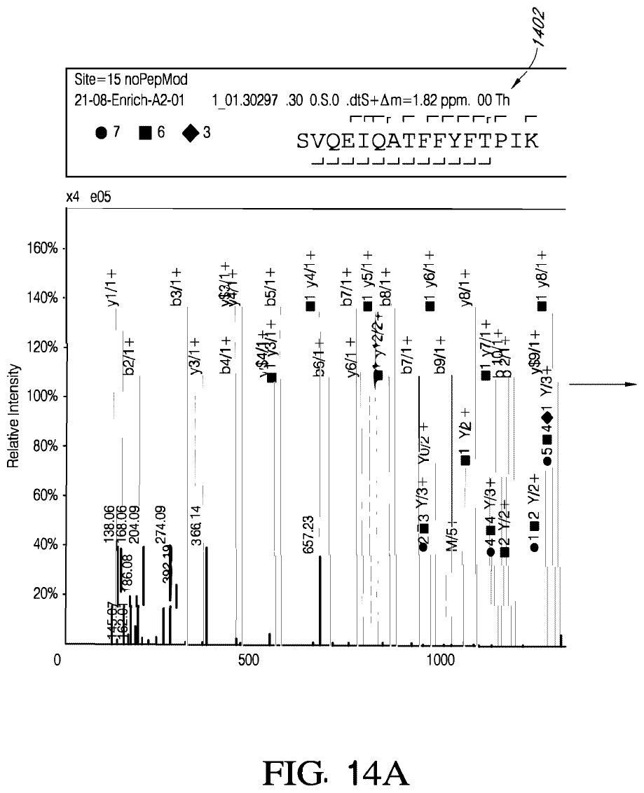

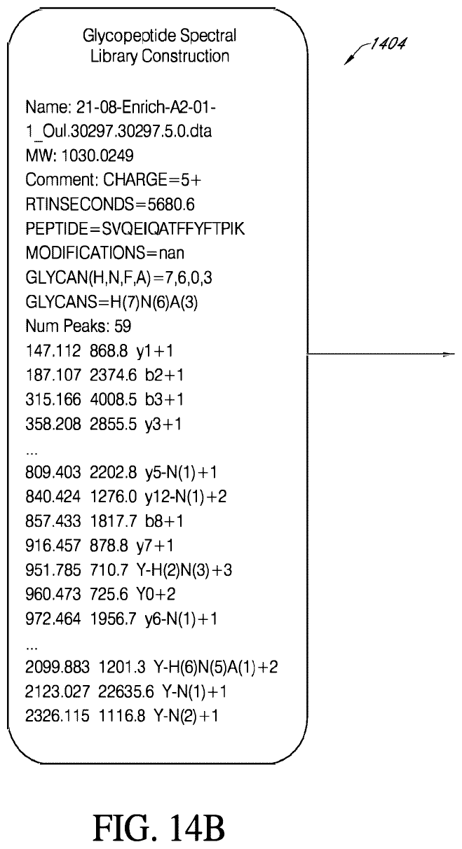

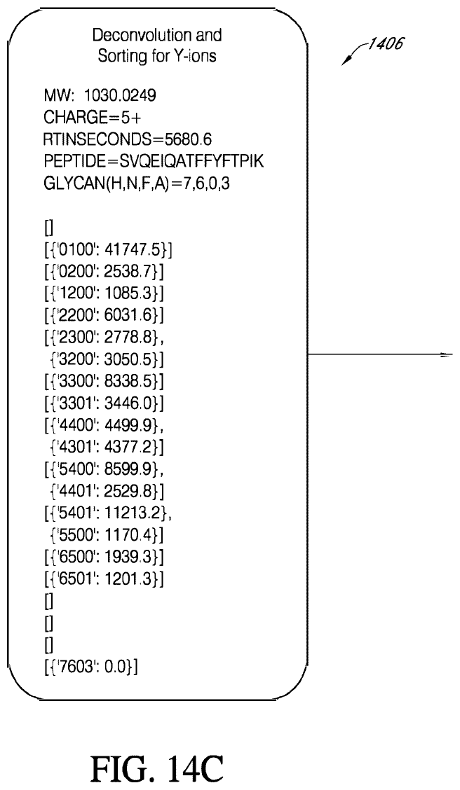

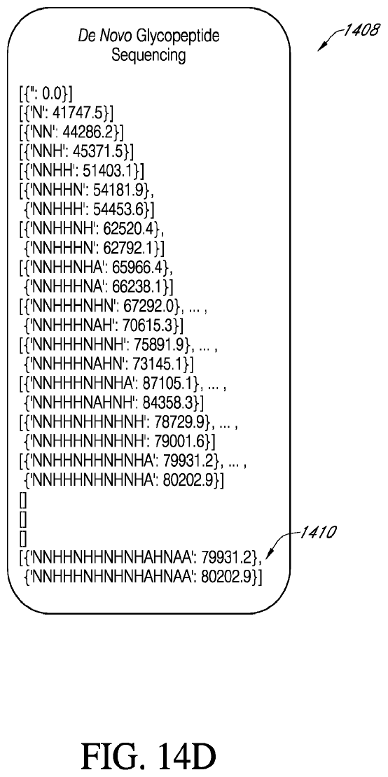

FIG. 4 is a block diagram of a computer system in accordance with various embodiments. Computer system 400 may be an example of one implementation for computing platform 302 described above in FIG. 3.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT), liquid crystal display (LCD), or light emitting diode (LED) for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

### VI. Exemplary Methodologies Relating to Glycopeptide Sequencing

### VI.A. Exemplary Methodology for Training a Machine Learning Model

In one of more embodiments, a method of using or training a machine learning model is described. The method may be implemented using, for example, at least a portion of workflow 100 as described in FIGS. 1, 2A, 2B, and/or 2C and/or using analysis system 300 as described in FIG. 3.

In accordance with one or more embodiments, a disclosed method may include receiving tandem mass spectral data for a plurality of fragments of a glycopeptide. The method may also include generating glycan fragment composition data for the glycopeptide using the tandem mass spectral data, the glycan fragment composition data identifying a plurality of glycan and glycopeptide compositions, and a plurality of total intensities for a plurality of glycan fragments identified from the plurality of fragments. In one or more embodiments, the method may include analyzing the glycan fragment composition data, via a machine learning model, to generate a prediction of a glycopeptide spectral library for the glycopeptide. The method may include predicting glycan structures of the glycopeptide, in one or more embodiments.

The glycopeptide spectral library identifies, for example, without limitation, a peptide sequence for the glycopeptide structure; an observed retention time for the glycopeptide structure; an observed glycan composition for the glycopeptide structure; a mass, a charge, and an intensity for each fragment of the plurality of fragments of the glycopeptide structure; and a glycan composition for each fragment of the plurality of fragments of the glycopeptide structure that represents at least a portion of a glycan. The plurality of fragments may include, for example, without limitation, a plurality of y ions, a plurality of b ions, and a plurality of Y ions. The fragments that represent at least a portion of a glycan are Y ion (or Y-ions) fragments. A Y ion is a glycan fragment in which the glycan (or portion thereof) is attached to the peptide sequence (or portion thereof). The plurality of fragments may include two or more fragments that have a same glycan composition with different charges. In one or more embodiments, the glycopeptide spectral library lists the plurality of fragments in increasing order with respect to mass. In other embodiments, the plurality of fragments included in the glycopeptide spectral library includes only Y ions or includes Y ions and either y ions or b ions.

In various embodiments, the method may include performing de novo interpretation of the predicted glycan structures of the intact glycopeptide using the predicted glycopeptide spectral library. In one or more embodiments, the machine learning model may be trained using a training input, wherein the training input is formed using a linear glycan sequence constructed using glycan composition data that identify a plurality of composition codes and a plurality of total intensities for a plurality of glycan fragments identified from spectral data of a plurality of glycopeptides.

In one or more embodiments, the spectral data may include mass spectrometry data obtained from an untargeted mass spectrometry system and wherein the mass spectrometry data include an observed retention time for the glycopeptide structure and a mass and an intensity for each fragment of the plurality of fragments of the glycopeptide structure.

In various embodiments, the method may include transferring a learning of the machine learning model to a new machine learning model to predict a new fragmentation pattern and a new retention time for a second glycopeptide structure.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1. D-VA: Prediction and Interpretation of Intact N-Glycopeptide Tandem Mass Spectra by Deep Learning

A dedicated in-house LC-MS system for large-scale glycopeptide spectral acquisition and data analysis (“D-VA”) was developed and its performance for de novo interpretation of N-glycan structures was benchmarked on intact glycopeptides. The method enables determination of detailed glycan and glycopeptide composition in human and mouse serum, and mouse tissue. Owing to the database-independent glycan mapping strategy and de novo definition of glycan compositions, D-VA can facilitate the identification of rare/new glycan structures. D-VA contains pre-trained models for predicting glycopeptide spectrum. It makes extensive use of transfer learning, which drastically reducES the amount of training data required.

RAW MS data of over 1,000 hours of LC-MS acquisition on human serum/plasma was used on the initial dataset for the development of deep learning models. A high-quality glycopeptide library was generated with over 1 million MS/MS spectra (post identification and quality control), corresponding to over 4,000 N-glycopeptides and 200 glycoproteins in human serum/plasma. Several dedicated quality control methods were specifically designed for glycopeptides, including feature detection post identification filter, picked strategy for large-scale glycoproteomics dataset.

Extensive fragmentation behavior was observed in our dataset. Compared to existing human serum glycopeptide library, the in-house library shows 20%˜50% more coverage on both peptide and glycan fragmentation (the prevalence of b/y ions and Y ions in glycopeptide MS/MS spectra). The improvement is achieved by optimization of MS acquisition parameter and data analytical pipeline that fully utilizes the MS/MS fragmentation information.

With the D-Va pre-trained models available for assigning glycan structures to MS2 glycopeptide spectra, the MS2 model was benchmarked against datasets of human tryptic treated glycopeptides. The training and testing data were collected from instruments across multiple collisional energies. Using these pre-trained models and specifically designed data structures, the prediction of a spectral library with MS2 intensities for a human glycopeptide (Test-Human-N-SCE dataset) with 13,239 glycopeptides, 15,849 glycopeptides with multiple charge states took approx. 14 hrs on a single.

For the transfer learning, the pre-trained model was applied to publicly accessible pGlyco mouse tissue data in order to test the robustness of our D-Va model based on a cross-species sample, and to examine whether the model is still highly accurate even with a small training dataset.

What is claimed is:

1. A method, comprising:
receiving tandem mass spectral data for a plurality of fragments of a glycopeptide;
generating glycan fragment composition data for the glycopeptide using the tandem mass spectral data, the glycan fragment composition data identifying a plurality of glycan and glycopeptide compositions, and a plurality of total intensities for a plurality of glycan fragments identified from the plurality of fragments;
analyzing the glycan fragment composition data, via a machine learning model, to generate a prediction of a glycopeptide spectral library for the glycopeptide; and
predicting glycan structures of the glycopeptide.

2. The method of claim 1, further comprising:
performing de novo interpretation of the predicted glycan structures of the intact glycopeptide using the predicted glycopeptide spectral library.

3. The method of claim 1 or 2, wherein the machine learning model is trained using a training input, wherein the training input is formed using a linear glycan sequence constructed using glycan composition data that identify a plurality of composition codes and a plurality of total intensities for a plurality of glycan fragments identified from spectral data of a plurality of glycopeptides.

4. The method of claim 3, wherein the spectral data comprises mass spectrometry data obtained from an untargeted mass spectrometry system and wherein the mass spectrometry data include an observed retention time for the glycopeptide structure and a mass and an intensity for each fragment of the plurality of fragments of the glycopeptide structure.

5. The method of claims 1-4, further comprising:
transferring a learning of the machine learning model to a new machine learning model to predict a new fragmentation pattern and a new retention time for a second glycopeptide structure.

6. A method for training a machine learning model to predict glycopeptide fragmentation patterns and retention times, the method comprising:
receiving spectral data for a plurality of fragments of a glycopeptide structure;
generating glycan fragment composition data using the spectral data, the glycan fragment composition data identifying a plurality of composition codes and a plurality of total intensities for a plurality of glycan fragments identified from the plurality of fragments using the spectral data;
creating a linear glycan sequence using the glycan fragment composition data;
forming a training input for a machine learning model using the linear glycan sequence;
training the machine learning model using the training input to predict a fragmentation pattern and a retention time for the glycopeptide structure.

7. The method of claim 6, wherein the spectral data comprises mass spectrometry data obtained from an untargeted mass spectrometry system and wherein the mass spectrometry data includes an observed retention time for the glycopeptide structure and a mass and an intensity for each fragment of the plurality of fragments of the glycopeptide structure.

8. The method of claim 7, further comprising:
generating a glycopeptide spectral library for the plurality of fragments of the glycopeptide structure using the mass spectrometry data, wherein the glycopeptide spectral library identifies:
an identified peptide sequence for the glycopeptide structure;
an observed retention time for the glycopeptide structure;
an identified glycan composition for the glycopeptide structure;
a mass, a charge, and an intensity for each fragment of the plurality of fragments of the glycopeptide structure; and
a glycan composition for each fragment of the plurality of fragments of the glycopeptide structure that represents at least a portion of a glycan.

9. The method of claim 6, wherein the spectral data comprises a glycopeptide spectral library for the plurality of fragments of the glycopeptide structure, the glycopeptide spectral library identifying:
an identified peptide sequence for the glycopeptide structure;
an observed retention time for the glycopeptide structure;
an identified glycan composition for the glycopeptide structure;
a mass, a charge, and an intensity for each fragment of the plurality of fragments of the glycopeptide structure; and
a glycan composition for each fragment of the plurality of fragments of the glycopeptide structure that represents at least a portion of a glycan.

10. The method of claim 8 or claim 9, wherein the glycopeptide spectral library lists the plurality of fragments in increasing order with respect to mass.

11. The method of any one of claims 6-10, wherein generating the glycan fragment composition data comprises:
identifying the plurality of glycan fragments from the plurality of fragments;
generating a composition code for each of the plurality of glycan fragments to form the plurality of composition codes;
generating a total intensity for each of the plurality of glycan fragments to form the plurality of total intensities.

12. The method of claim 11, wherein generating the total intensity comprises:
combining, for a glycan fragment of the plurality of fragments, intensities for any fragments of the plurality of fragments that have a same glycan composition.

13. The method of claim 11 or claim 12, wherein the plurality of composition codes and the plurality of total intensities is presented in the glycan fragment composition data as a list ordered by increasing order of total number of molecules.

14. The method of any one of claims 6-13, wherein a glycan fragment of the plurality of glycan fragments represents all fragments of the plurality of fragments that have a same glycan composition.

15. The method of any one of claims 6-14, wherein creating the linear glycan sequence comprises:
converting, for each corresponding glycan fragment of the plurality of glycan fragments, a composition code of the plurality of composition codes for the corresponding glycan fragment into a linear fragment sequence to form a plurality of linear fragment sequences;
computing, for each corresponding glycan fragment of the plurality of glycan fragments a cumulative intensity for the linear fragment sequence to form a plurality of cumulative intensities, the cumulative intensity being a sum of a total intensity of the plurality of total intensities for the glycan fragment and, if present, a previously computed intensity for a previously generated linear fragment sequence.

16. The method of claim 15, wherein creating the linear glycan sequence further comprises:
identifying a set of linear fragment sequences from the plurality of linear fragment sequences having a longest molecule length; and
selecting, from the set of linear fragment sequences, one linear fragment sequence having a maximum cumulative intensity as the linear glycan sequence.

17. The method of any one of claims 6-16, wherein the plurality of fragments comprises a plurality of y ions, a plurality of b ions, and a plurality of Y ions.

18. The method of any one of claims 6-17, wherein the plurality of glycan fragments corresponds to a plurality of Y ions of the plurality of fragments.

19. The method of any one of claims 6-18, wherein the plurality of fragments includes two fragments that have a same glycan composition.

20. The method of any one of claims 6-19, wherein forming the training input comprises:
forming the training input for the machine learning model using the linear glycan sequence, a peptide sequence for the glycopeptide structure, and one-hot encoding.

21. The method of any one of claims 6-20, wherein forming the training input comprises:
discretizing at least a portion of the spectral data to form the training input.

22. The method of any one of claims 6-21, wherein the machine learning model comprises a recurrent neural network.

23. The method of any one of claims 6-20, wherein the fragmentation pattern includes at least one of a set of m/z ratios for the glycopeptide structure or a set of intensities for the glycopeptide structure.

24. The method of any one of claims 6-22, wherein the retention time for the glycopeptide structure is an index retention time (iRT).

25. The method of claim 6, wherein the linear glycan sequence is one of a plurality of linear glycan sequences used to form the training input for the machine learning model.

26. A method comprising:
forming a training input for a machine learning model using a linear glycan sequence created for a glycopeptide structure,
wherein the linear glycan sequence is constructed using glycan composition data that identifies a plurality of composition codes and a plurality of total intensities for a plurality of glycan fragments identified from spectral data;
training the machine learning model using the training input; and
predicting a fragmentation pattern and a retention time for the glycopeptide structure using the trained machine learning model.

27. The method of claim 26, wherein the spectral data comprises mass spectrometry data obtained from an untargeted mass spectrometry system for a plurality of fragments of the glycopeptide structure and wherein the mass spectrometry data includes an observed retention time for the glycopeptide structure and a mass and an intensity for each fragment of the plurality of fragments.

28. The method of claim 27, further comprising:
generating a glycopeptide spectral library for the plurality of fragments of the glycopeptide structure using the mass spectrometry data, wherein the glycopeptide spectral library identifies:
an identified peptide sequence for the glycopeptide structure;
an observed retention time for the glycopeptide structure;
an identified glycan composition for the glycopeptide structure;
a mass, a charge, and an intensity for each fragment of the plurality of fragments of the glycopeptide structure; and
a glycan composition for each fragment of the plurality of fragments of the glycopeptide structure that represents at least a portion of a glycan.

29. The method of claim 26, wherein the spectral data comprises a glycopeptide spectral library for a plurality of fragments of the glycopeptide structure, the glycopeptide spectral library identifying:
an identified peptide sequence for the glycopeptide structure;
an observed retention time for the glycopeptide structure;
an identified glycan composition for the glycopeptide structure;
a mass, a charge, and an intensity for each fragment of the plurality of fragments of the glycopeptide structure; and
a glycan composition for each fragment of the plurality of fragments of the glycopeptide structure that represents at least a portion of a glycan.

30. The method of claim 28 or claim 29, wherein the glycopeptide spectral library lists the plurality of fragments in increasing order with respect to mass.

31. The method of any one of claims 26-30, further comprising:
identifying a plurality of glycan fragments from a plurality of fragments identified in the spectral data;
generating the glycan fragment composition data in which a composition code and a total intensity is generated for each glycan fragment of the plurality of glycan fragments.

32. The method of claim 31, wherein the total intensity for a glycan fragment is generated by combining intensities for any fragments of the plurality of fragments that have a same glycan composition.

33. The method of claim 31 or claim 32, wherein the plurality of composition codes and the plurality of total intensities is presented in the glycan fragment composition data as a list ordered by increasing order of total number of molecules.

34. The method of any one of claims 26-33, wherein a glycan fragment of the plurality of glycan fragments represents all fragments of a plurality of fragments identified in the spectral data that have a same glycan composition.

35. The method of any one of claims 26-34, further comprising:
creating the linear glycan sequence, wherein the creating comprises:
converting, for each corresponding glycan fragment of the plurality of glycan fragments, a composition code of the plurality of composition codes for the corresponding glycan fragment into a linear fragment sequence to form a plurality of linear fragment sequences;
computing, for each corresponding glycan fragment of the plurality of glycan fragments a cumulative intensity for the linear fragment sequence to form a plurality of cumulative intensities, the cumulative intensity being a sum of a total intensity of the plurality of total intensities for the glycan fragment and, if present, a previously computed intensity for a previously generated linear fragment sequence.

36. The method of claim 35, wherein creating the linear glycan sequence further comprises:
identifying a set of linear fragment sequences from the plurality of linear fragment sequences having a longest molecule length; and
selecting, from the set of linear fragment sequences, one linear fragment sequence having a maximum cumulative intensity has the linear glycan sequence.

37. The method of any one of claims 26-36, wherein the spectral data identifies a plurality of fragments that include a plurality of y ions, a plurality of b ions, and a plurality of Y ions.

38. The method of any one of claims 26-37, wherein the plurality of glycan fragments corresponds to a plurality of Y ions.

39. The method of any one of claims 26-38, wherein the spectral data identifies a plurality of fragments that includes two fragments that have a same glycan composition.

40. The method of any one of claims 26-39, wherein forming the training input comprises:
forming the training input for the machine learning model using the linear glycan sequence, a peptide sequence for the glycopeptide structure, and one-hot encoding.

41. The method of any one of claims 26-40, wherein forming the training input comprises:
discretizing at least a portion of the spectral data to form the training input.

42. The method of any one of claims 26-41, wherein the machine learning model comprises a recurrent neural network.

43. The method of any one of claims 26-40, wherein the fragmentation pattern includes at least one of a set of m/z ratios for the glycopeptide structure or a set of intensities for the glycopeptide structure.

44. The method of any one of claims 26-42, wherein the retention time for the glycopeptide structure is an index retention time (iRT).

45. The method of any one of claims 26-44, further comprising:
augmenting a glycoproteomic database using the fragmentation pattern and the retention time predicted for the glycopeptide structure using the trained machine learning model.

46. The method of claim 45, further comprising:
performing untargeted mass spectrometry on a sample to detect an observed fragmentation pattern and an observed retention time; and
matching the observed fragmentation pattern and the observed retention time to the glycopeptide structure using the augmented glycoproteomic database.

47. The method of any one of claims 26-44, further comprising:
augmenting a library of information for N-linked glycopeptide structures with the fragmentation pattern and the retention time predicted for the glycopeptide structure using the trained machine learning model; and
matching an observed fragmentation pattern and an observed retention time to the glycopeptide structure using the augmented library of information.

48. The method of any one of claims 26-47, further comprising:
generating a multiple reaction monitoring-mass spectrometry (MRM-MS) panel based on the fragmentation pattern and the retention time predicted for the glycopeptide structure; and
performing a targeted MRM-MS run based on the MRM-MS panel.

49. The method of any one of claims 26-47, further comprising:
generating a panel for a DIA single shot mass spectrometry system based on the fragmentation pattern and the retention time predicted for the glycopeptide structure; and
performing a DIA single shot run based on the panel.

50. The method of any one of claims 26-49,
confirming a detection of the glycopeptide structure via mass spectrometry using the fragmentation pattern and the retention time predicted for the glycopeptide structure.

51. The method of any one of claims 26-50, wherein the glycopeptide structure is an N-linked glycopeptide structure and further comprising:
transferring learning of the machine learning model to a new machine learning model to predict a new fragmentation pattern and a new retention time for an O-linked glycopeptide structure.

52. A method comprising:
training a machine learning model to predict fragmentation patterns and retention times for a plurality of glycopeptide structures using a plurality of linear glycan sequences constructed for the plurality of glycopeptide structures;
wherein a linear glycan sequence of the plurality of linear glycan sequences is constructed using glycan composition data that identifies a plurality of composition codes and a plurality of total intensities for a plurality of glycan fragments identified from spectral data;
augmenting a glycoproteomic database using the fragmentation patterns and the retention time predicted for the plurality of glycopeptide structures using the trained machine learning model.

53. The method of claim 52, further comprising:
performing untargeted mass spectrometry on a sample to detect an observed fragmentation pattern and an observed retention time; and
matching the observed fragmentation pattern and the observed retention time to a glycopeptide structure using the augmented glycoproteomic database.

54. The method of claim 52 or claim 53, wherein the fragmentation patterns include at least one of m/z ratios for the plurality of glycopeptide structures or intensities for the plurality of glycopeptide structures.

55. The method of any one of claims 52-54, wherein the retention times for the plurality of glycopeptide structures are index retention times (iRT).

56. A method comprising:
training a machine learning model to predict fragmentation patterns and retention times for a plurality of N-linked glycopeptide structures using a plurality of linear glycan sequences constructed for the plurality of N-linked glycopeptide structures;
wherein a linear glycan sequence of the plurality of linear glycan sequences is constructed using glycan composition data that identifies a plurality of composition codes and a plurality of total intensities for a plurality of glycan fragments identified from spectral data;
augmenting a library of information for the plurality of N-linked glycopeptide structures with the fragmentation patterns and the retention times predicted using the trained machine learning model; and
matching an observed fragmentation pattern and an observed retention time to an N-linked glycopeptide structure using the augmented library of information.

57. The method of claim 56, wherein the fragmentation patterns include at least one of m/z ratios for the plurality of N-linked glycopeptide structures or intensities for the plurality of N-linked glycopeptide structures.

58. The method of claim 56 or claim 57, wherein the retention times for the plurality of N-linked glycopeptide structures are index retention times (iRT).

59. A method comprising:
training a machine learning model to predict fragmentation patterns and retention times for a plurality of glycopeptide structures using a plurality of linear glycan sequences constructed for the plurality of glycopeptide structures,
wherein a linear glycan sequence of the plurality of linear glycan sequences is constructed using glycan composition data that identifies a plurality of composition codes and a plurality of total intensities for a plurality of glycan fragments identified from spectral data;
generating a multiple reaction monitoring-mass spectrometry (MRM-MS) panel based on the fragmentation pattern and the retention time predicted for at least one glycopeptide structure of the plurality of glycopeptide structures; and
performing a targeted MRM-MS run based on the MRM-MS panel.

60. The method of claim 59, wherein the fragmentation pattern includes a set of m/z ratios for the at least one glycopeptide structure or a set of intensities for the at least one glycopeptide structure.

61. The method of claim 59 or claim 60, wherein the retention time for the at least one glycopeptide structure is an index retention times (iRT).

62. A method comprising:
training a machine learning model to predict fragmentation patterns and retention times for a plurality of N-linked glycopeptide structures using a plurality of linear glycan sequences constructed for the plurality of N-linked glycopeptide structures;
wherein a linear glycan sequence of the plurality of linear glycan sequences is constructed using glycan composition data that identifies a plurality of composition codes and a plurality of total intensities for a plurality of glycan fragments identified from spectral data;
transferring learning of the machine learning model to a new machine learning model to predict a new fragmentation pattern and a new retention time for an O-linked glycopeptide structure.

63. The method of claim 62, wherein the new fragmentation pattern includes a set of m/z ratios for the O-linked glycopeptide structure or a set of intensities for the O-linked glycopeptide structure.

64. The method of claim 62 or claim 63, wherein the retention time for the O-linked glycopeptide structure is an index retention times (iRT).

65. A method comprising:
training a machine learning model to predict a fragmentation pattern and a retention time for a glycopeptide structure using a linear glycan sequence constructed for the glycopeptide structure,
wherein the linear glycan sequence is constructed using glycan composition data that identifies a plurality of composition codes and a plurality of total intensities for a plurality of glycan fragments identified from spectral data for the glycopeptide structure;
selecting an m/z interrogation range for DIA single shot mass spectrometry based on the fragmentation pattern and the retention time predicted for the glycopeptide structure; and
performing a DIA single shot mass spectrometry run based on the selected m/z range.

66. The method of claim 65, wherein the fragmentation pattern includes a set of m/z ratios for the at least one glycopeptide structure or a set of intensities for the at least one glycopeptide structure.

67. The method of claim 65 or claim 66, wherein the retention time for the at least one glycopeptide structure is an index retention times (iRT).

68. A system comprising:
a data processor; and
a non-transitory computer readable storage medium containing instructions which, when executed on the data processor, cause the data processor to perform part or all of the method of any one of claims 6-68.

69. A computer-program product tangibly embodied in a non-transitory machine-readable storage medium, the computer-program product including instructions configured to cause a data processor to perform part or all of the method of any one of claims 6-68.
